(19) United States
(12) Patent Application Publication
(10) Pub. No.: US 2024/0225861 A1
(43) Pub. Date: Jul. 11, 2024

(54) PROSTHETIC HAND DEVICE USING A WEARABLE ULTRASOUND MODULE AS A HUMAN MACHINE INTERFACE

(71) Applicant: THE HONG KONG POLYTECHNIC UNIVERSITY, Hong Kong (CN)

(72) Inventors: Vaheh NAZARI, Hong Kong (CN); Yongping ZHENG, Hong Kong (CN); Tsung Kwan SHEA, Hong Kong (CN)

(21) Appl. No.: 18/305,415

(22) Filed: Apr. 24, 2023

Related U.S. Application Data
(60) Provisional application No. 63/438,402, filed on Jan. 11, 2023.

(51) Int. Cl. A61F 2/70; A61F 2/58; G06T 7/246; G06V 10/82; G06V 40/20

(52) U.S. Cl. CPC A61F 2/70; A61F 2/585; A61F 2/586; G06T 7/246; G06V 10/82; G06V 40/20; A61F 2002/5093; A61F 2002/587; A61F 2002/704

(57) ABSTRACT

A prosthetic hand device mountable on a residual limb of an amputee is provided. The prosthetic hand device includes a myoelectric hand having five mechanical fingers actuatable to provide multiple degrees of freedom of movement, a control assembly including an ultrasound module as a human-machine interface, wherein the ultrasound module is configured to acquire ultrasound images of a region of the residual limb, a transfer learning model having a convolutional neural network architecture for obtaining extracted features from the ultrasound images, and an artificial intelligence model executed by one or more processors and configured to classify the extracted features from the ultrasound images for determining a volitional movement of the amputee in real-time. The volitional movement is transmitted to the myoelectric hand to dynamically and proportionally control the five mechanical fingers based on at least the volitional movement.

### CROSS REFERENCE TO RELATED APPLICATIONS

This application claims the benefit of U.S. Provisional Patent Application No. 63/438,402 filed on Jan. 11, 2023, the disclosure of which is incorporated by reference herein in its entirety.

### FIELD OF THE INVENTION

The present disclosure generally relates to a myoelectric prosthetic device. In particular, the present disclosure relates to a non-invasive and precise technique for controlling multiple degrees of freedom prosthetic hand device or other exoskeleton devices.

### BACKGROUND OF THE INVENTION

The field of human machine interfaces (HMIs) and wearable technology is rapidly growing, driven by its potential to bring significant benefits to patients in healthcare and rehabilitation settings. HMIs can improve mobility and reduce dependence on human assistance, leading to improved quality of life for patients. Several types of prosthetic control systems for prostheses and exoskeletons utilizing HMI are being proposed and developed, but they face numerous challenges. Despite significant progress in mechanical design, there are still many issues to be addressed at higher levels of the HMI control hierarchy.

Particularly, significant amount of works are needed to overcome the challenges of accurately and efficiently identifying a user's motion intention. This requires addressing the complex interplay between the patient's intent, the physical environment, and the limitations of the HMI system. Despite the numerous studies on various HMIs, there remains a persistent lack of proportional and reliable control over prosthetics with multiple degrees of freedom.

Conventional methods use the biological signals continuously, which may be recorded by a number of sensors and electrodes when interfacing with either the peripheral nervous system (PNS) or the central nervous system (CNS), for controlling the exoskeleton device. These methods can be categorized as either non-invasive or invasive. Some examples of the non-invasive methods include surface electromyography (sEMG), electroencephalography (EEG), forcemyography (FMG), mechanomyography (MMG), magnetoencephalography (MEG), force sensitive resistance (FSR). Invasive methods include implanted electromyography (iEMGs), myoelectric implantable recording arrays (MIRAs), electroneurography (ENG), electrocorticography (ECoG), brain chip interfaces (BCHIs), and magnetomicrometry (MM).

Recently, the field of prosthetics has seen increased efforts in creating state-of-the-art devices that are intuitive and easy to control. By monitoring the user's intentions, these new prostheses aim to enhance the user's experience without disrupting their normal activities. Over the past decade, researchers have focused on developing non-invasive methods for capturing the user's intentions, such as placing electrodes on the scalp or skeletal muscles.

To increase the effectiveness of these electrodes, conductive gel is usually used to increase the contact area and conductivity. Bipolar electrodes are placed on skeletal muscles to record muscular activity and capture low-amplitude electrical impulses. However, there are many variables that can alter the data received by these sensors, such as electrode placement and movement, sweat, and even electronic noise. Additionally, the spatial resolution of these techniques is limited due to signal interference from nearby or overlapping muscles.

Despite these challenges, researchers continue to work on using sEMG to control prostheses and exoskeletons with several degrees of freedom. However, this method is limited as it cannot accurately capture the activity of deep muscles. Another disadvantage is the time and effort required to train individuals to control robots using biological signals, as the relationships between these signals and the force or angle generated by the muscles are not always linear.

Biomaterials have a long history of use in implantation procedures. Cutting-edge tools and approaches such as implanted myoelectric sensors, peripheral nerve implants, targeted muscle reinnervation, brain computer interfaces, and implanted stimulators have the potential to revolutionize the field of neuroscience and offer new ways for discovery. Some invasive methods involve the implantation of electrodes or other devices into the patient's brain, spinal cord, or muscles. These implants can detect and transmit electrical activity generated by nerve or muscle activity, and provide a direct link between the brain, nerves, and muscles. This can facilitate communication between neurons and computers, or vice versa. These invasive methods aim to improve the quality of signals recorded from sensors and decrease noise, provide more consistent biological signals, and offer a more precise understanding of brain and muscle activities. However, they also raise concerns about the safety and efficacy of surgical procedures and implanted devices due to the need for electrodes to be implanted inside the body. Additionally, these signals can still be subject to noise, just as with non-invasive methods.

The most common application of these methods is in prostheses with a single degree of freedom. To effectively analyze and classify biological signals, sophisticated characteristic algorithms are necessary. These algorithms should be able to accurately identify the various signals collected with minimal error. Recently, there have been many advancements in the processing and classification of biological signals, thanks to the use of various machine learning and deep learning techniques. For example, machine learning has been successful in achieving high performance accuracy in a wide range of fields. Many signal classification algorithms, including K nearest neighbours (KNN), Support Vector Machines (SVM), Principal Component Analysis (PCA), Linear Discriminant Analysis (LDA), Artificial Neural Networks (ANN), Convolutional Neural Networks (CNN), and Bayes networks, can be utilized to improve robot control with an accuracy of around 90%.

The use of machine learning and deep learning techniques has achieved significant progress in the analysis and categorization of biological signals. However, the application in prostheses and exoskeleton devices is not without challenges. These algorithms require high quality data for optimizing the accuracy. The training data may not be transferrable and the model may not be the same for each patient. The analysis is computationally intensive, and any noise or interference may lead to incorrect classifications.

Accordingly, there is a need in the art to have a non-invasive and precise technique for controlling multiple degrees of freedom prostheses and exoskeleton devices. Furthermore, other desirable features and characteristics will become apparent from the subsequent detailed description and the appended claims, taken in conjunction with the accompanying drawings and this background of the disclosure.

### SUMMARY OF THE INVENTION

Provided herein is a non-invasive method for controlling prosthetic hand device or other exoskeleton devices with multiple degrees of freedom. It is the objective of the present disclosure to achieve high precision control using wearable ultrasound device as a human machine interface.

In certain aspects of the present disclosure, a prosthetic hand device mountable on a residual limb of an amputee is provided. The prosthetic hand device includes a myoelectric hand having five mechanical fingers actuatable to provide multiple degrees of freedom of movement, a control assembly including an ultrasound module as a human-machine interface (HMI), wherein the ultrasound module is configured to acquire ultrasound images of a region of the residual limb, a transfer learning model having a convolutional neural network (CNN) architecture for obtaining extracted features from the ultrasound images, and an artificial intelligence (AI) model executed by one or more processors and configured to classify the extracted features from the ultrasound images for determining a volitional movement of the amputee in real-time. The volitional movement is transmitted to the myoelectric hand to dynamically and proportionally control the five mechanical fingers based on at least the volitional movement. The ultrasound module is configured to capture the ultrasound images of flexor digitorum superficialis (FDS), flexor digitorum profundus (FDP), and flexor pollicis longus (FPL) muscles for determining the volitional movement of the amputee.

In an embodiment, the prosthetic hand device further includes a proportional control mechanism enabling the amputee to dynamically control a speed of finger flexion and an angle of finger flexion. The proportional control mechanism is configured to monitor a degree of muscular contraction using the ultrasound images for predicting a proportional change.

In an embodiment, the transfer learning model further includes a plurality of convolutional layers, a flatten layer, and a fully connected layer.

In an embodiment, the ultrasound module includes an ultrasound transducer and a control circuit. The control circuit is configured to cause the ultrasound transducer to repeatedly and regularly generate acoustic waves which is directed into the residual limb of the amputee. The ultrasound transducer measures acoustic reflections for information to be used to generate the ultrasound images.

In an embodiment, the ultrasound module further includes a sticky silicone pad placed between head of the ultrasound transducer and the residual limb for enhancing image quality. The sticky silicone pad is prepared by mixing silicones with 00 hardness and 05 hardness in a 3:1 ratio.

In an embodiment, the myoclectric hand includes a machine learning model. The ultrasound images are separated into a training dataset and a validation dataset. The transfer learning model extracts features from the training dataset to obtain the extracted features for training the machine learning model. The validation dataset is utilized to evaluate an accuracy of the AI model.

In an embodiment, the machine learning model includes one or more machine learning algorithms selected from the group consisting of random forest (RF), k-nearest neighbors classifier (KNN), and support vector machine (SVM).

In an embodiment, the CNN architecture is selected from the group consisting of VGG16, VGG19, and Inceprion-Res-Net-V2.

In an embodiment, the myoelectric hand includes an actuating system to provide multiple degrees of freedom, wherein the actuating system includes plural artificial metacarpophalangcal (MCP) joints at the five mechanical fingers, an additional MCP joint at the first mechanical finger, and plural artificial proximal interphalangeal (PIP) joints at the second to the fifth mechanical fingers, and wherein the additional MCP joint is rotatable about a second axis substantially orthogonal to a first axis of the MCP joint at the first mechanical finger to perform abduction and adduction.

In an embodiment, the myoelectric hand includes an artificial tendon and a control unit configured to actuate the artificial tendon to flex and extend an individual mechanical finger. The control unit comprises a motor, a motor shaft, a roller, and a tension spring. The artificial tendon is attached to the tension spring at a first end, through a fingertip of the individual mechanical finger to the roller at a second end. The motor is powered to cause the motor shaft and the roller to rotate to drive a pulling movement of the individual mechanical finger via the artificial tendon to cause the individual mechanical finger to flex or adduct. The tension spring stores energy from flexion and releases the energy when the motor is driven in an opposite direction to cause the individual mechanical finger to extend or abduct.

In an embodiment, the control assembly is attached on a socket having a shape based on a normal human hand. The actuating system further includes a wrist rotational joint provided between the socket and the myoelectric hand, wherein the prosthetic hand device includes an A-mode ultrasound transducer arranged to capture ultrasound images for determining an intended wrist movement and controlling the wrist rotational joint.

In an embodiment, the myoelectric hand includes a base portion connected to and provides support to the five mechanical fingers. Each of the five mechanical fingers and the base portion are made of a base material and silicone. The base material is selected from the group of materials consisting of nylon, plastic, polypropylene (PP), Acrylonitrile Butadiene Styrene (ABS), and vinyl. The silicone has a frictional gripping characteristic with a shore hardness value of 00-50.

In an embodiment, the myoelectric hand includes a sensory feedback mechanism having plural force sensors. Each of the five mechanical fingers comprises a silicone layer at a fingertip region, and the force sensor is mounted in the fingertip region under the silicone layer. The five mechanical fingers are dynamically and individually actuated by a control unit, which is controlled by a microprocessor based on the ultrasound images and output voltages of the force sensors.

In an embodiment, the sensory feedback mechanism is configured to stimulating different nerves of the amputee with different amplitudes and frequencies to allow the amputee to dynamically control a degree of flexion of each of the five mechanical fingers, and decrease a phantom pain.

In an embodiment, a curved surface part, made of black nylon material, ABS, or PP, is fixed under the silicone layer for transferring force to the force sensor.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects and advantages of the present invention are disclosed as illustrated by the embodiments hereinafter.

### BRIEF DESCRIPTION OF THE DRAWINGS

The appended drawings contain figures to further illustrate and clarify the above and other aspects, advantages, and features of the present disclosure. It will be appreciated that these drawings depict only certain embodiments of the present disclosure and are not intended to limit its scope. It will also be appreciated that these drawings are illustrated for simplicity and clarity and have not necessarily been depicted to scale. The present disclosure will now be described and explained with additional specificity and detail through the use of the accompanying drawings in which:

FIG. 1 is the overall schematic of the method for controlling a prosthetic hand device using an ultrasound probe in accordance with the present disclosure;

FIG. 2 is the arrangement for capturing the muscle activities of each participant;

FIG. 3 is the position of the mounting position of the ultrasound transducer;

FIG. 4A shows the B-Mode ultrasound images captured from muscle activities during performing different hand gestures;

FIG. 4B shows the ultrasound images captured from muscle activities for 0%, 50%, and 100% muscle contraction;

FIG. 5 shows the comparison of the B-Mode ultrasound images captured with sticky silicone pad and ultrasound gel;

FIG. 6 is the overall schematic of the classification process for extracting features to train a model that can classify different hand gestures;

FIG. 7 is a perspective view of the prosthetic hand device in accordance with certain embodiments of the present disclosure;

FIG. 8 is a side internal view of the prosthetic hand device of FIG. 7;

FIG. 9 is an exploded view of the prosthetic hand device of FIG. 7;

FIG. 10 is a first exploded view of the myoelectric hand in accordance with certain embodiments of the present disclosure;

FIG. 11 shows the mechanical joints in the myoelectric hand in accordance with certain embodiments of the present disclosure;

FIG. 12 is a second exploded view of the myoelectric hand in accordance with certain embodiments of the present disclosure;

FIG. 13 shows the artificial tendon fixed to the motor of the control unit in accordance with certain embodiments of the present disclosure;

FIG. 14 shows the artificial tendon and the control unit for controlling the mechanical fingers of the myoelectric hand in accordance with certain embodiments of the present disclosure;

FIG. 15 is the internal structure of the mechanical finger in accordance with certain embodiments of the present disclosure;

FIG. 16 is a circuit diagram of the fingertip force measurement unit in accordance with certain embodiments of the present disclosure; and

FIG. 17 is a graph showing the measured force by the force sensor mounted on the fingertip region with different value of load cell.

### DETAILED DESCRIPTION OF THE INVENTION

The following detailed description is merely exemplary in nature and is not intended to limit the disclosure or its application and/or uses. It should be appreciated that a vast number of variations exist. The detailed description will enable those of ordinary skilled in the art to implement an exemplary embodiment of the present disclosure without undue experimentation, and it is understood that various changes or modifications may be made in the function and structure described in the exemplary embodiment without departing from the scope of the present disclosure as set forth in the appended claims.

The benefits, advantages, solutions to problems, and any element(s) that may cause any benefit, advantage, or solution to occur or become more pronounced are not to be construed as a critical, required, or essential features or elements of any or all of the claims. The invention is defined solely by the appended claims including any amendments made during the pendency of this application and all equivalents of those claims as issued.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The terms “comprising,” “having,” and “including” or any other variation thereof, are to be construed as open-ended terms (i.e., meaning “including, but not limited to,”) unless otherwise noted. The use of any and all examples, or exemplary language (e.g., “such as”) provided herein, is intended merely to illuminate the invention better and does not pose a limitation on the scope of the invention unless otherwise claimed. No language in the specification should be construed as indicating any non-claimed element as essential to the practice of the invention. Further, unless expressly stated to the contrary, “or” refers to an inclusive “or” and not to an exclusive “or”. For example, a condition A or B is satisfied by any one of the following: A is true and B is false, A is false and B is true, and both A and B are true. Terms of approximation, such as “about”, “generally”, “approximately”, and “substantially” include values within ten percent greater or less than the stated value.

Unless otherwise defined, all terms (including technical and scientific terms) used in the embodiments of the present invention have the same meaning as commonly understood by an ordinary skilled person in the art to which the present invention belongs.

As used herein, the terms “coupled” or “connected,” or any variant thereof, covers any coupling or connection, either direct or indirect, between two or more elements, unless otherwise indicated or clearly contradicted by context.

In light of the background, it is desirable to provide machine learning and deep learning techniques for implementing a non-invasive and high precision control of the prostheses or exoskeletons with multiple degrees of freedom. In certain embodiments, the method is characterized in that the high precision control of the prosthetic hand device or the exoskeletons is achieved using a wearable ultrasound device as a human-machine interface (HMI). Furthermore, an ultrasound transducer is integrated into the prostheses for enabling real-time high precision control.

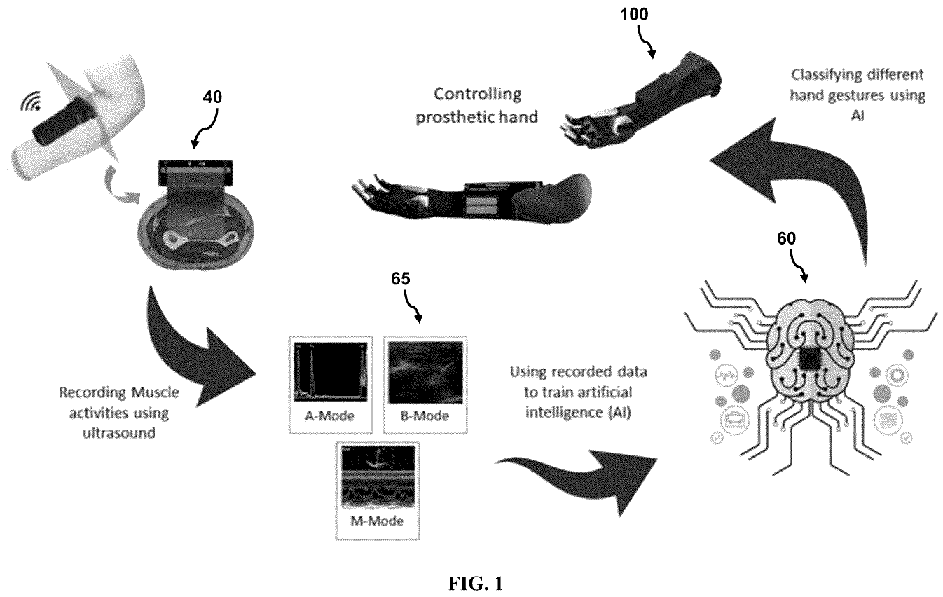

The first embodiment of the present disclosure is related to a prosthetic hand device 100 mountable on a residual limb of an amputee that can be controlled based on the volitional movement sensed from a residual limb. FIG. 1 shows for the overall schematic of the method used in one exemplary application of controlling a prosthetic hand device 100 for an amputee with the forearm amputated. It is one of the objectives of the present disclosure to replicate the normal kinematics of a hand and allow the amputee to control the prosthetic hand device 100 for performing various tasks. Unlike the conventional approaches of using surface electromyography (sEMG) sensors, the present disclosure uses an ultrasound transducer 40 to capture ultrasound images of the muscles for controlling the prosthetic hand device 100 as an HMI in real-time. The ultrasound imaging enables capturing the activities of deep and superficial muscles for determining the desired movement and intention of the user. Different ultrasound modes can be used to capture and provide muscle activity. There are five different ultrasound modes, but only three of them (A-mode, B-mode, and M-mode) can be used for controlling the prosthetic hand device 100. Previous research on controlling prostheses had a number of major limitations: the use of non-portable ultrasound probes and the lack of precision and agility. In particular, a wired ultrasound probe was employed to collect muscle activity, which resulted in discomfort for the user. The present disclosure overcomes this issue by utilizing a wireless ultrasound transducer 40 to send muscle activity data to a computer via Wi-Fi. The prosthetic control system comprises an artificial intelligence (AI) model 60, which can be executed by one or more processors to implement in software for precisely determining the prosthetic movement based on the volitional movement sensed. This prosthetic control system enables the development of a more comfortable and more accurate system for a prosthetic hand device 100 or other exoskeleton devices. In particular, by using a six-degree of freedom prosthetic hand device 100, which is designed based on human natural hand and anatomy, the one or more processors can control the prosthetic hand device 100 precisely using the patient's remaining muscular contractions or neuroactivities as input. The prosthetic control system is assessed on the hand functionality to evaluate the prosthetic hand device 100 and the AI model 60.

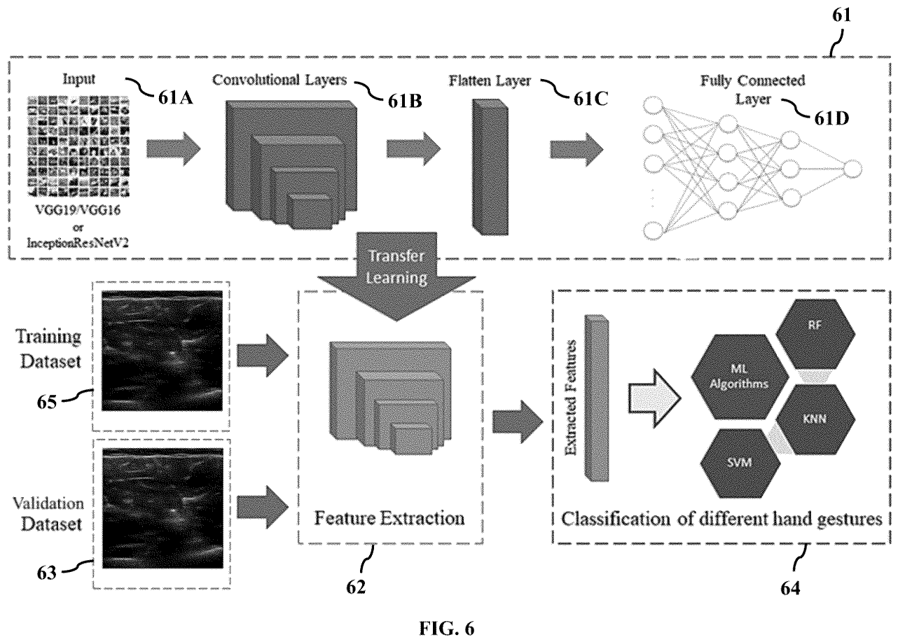

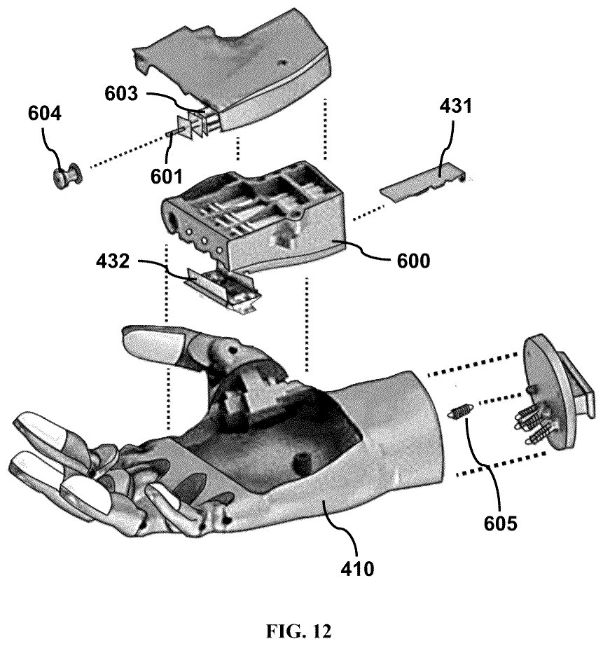

The ultrasound transducer 40 is used to record muscle activities for providing training datasets 65 of different hand gestures to train the AI model 60. A transfer learning model 61 (shown in FIG. 6) with a set of weights pre-trained on ImageNet is utilized to extract features from the ultrasound images of the training datasets 65. The AI model 60 can therefore classify the extract features from the ultrasound images for determining a volitional movement of the amputee in real-time, while the predicted volitional movement is transmitted to the microprocessor 432 (shown in FIG. 12) of the prosthetic hand device 100 to perform different hand gestures.

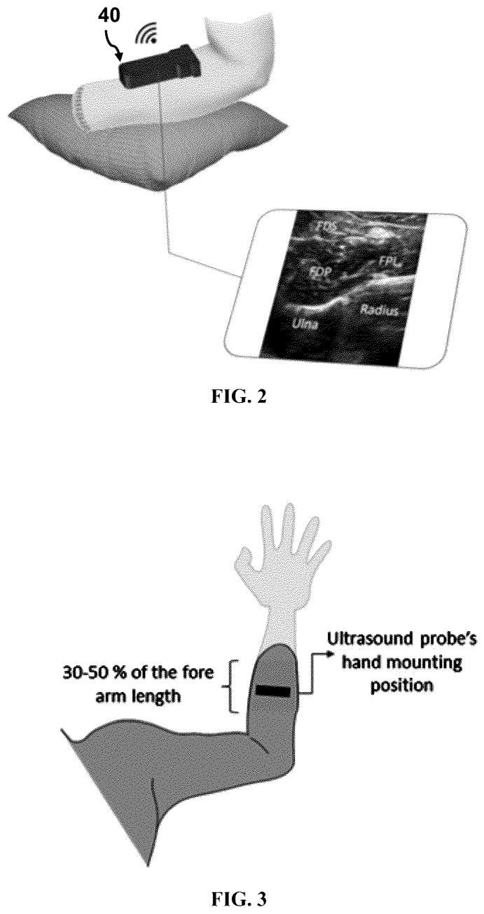

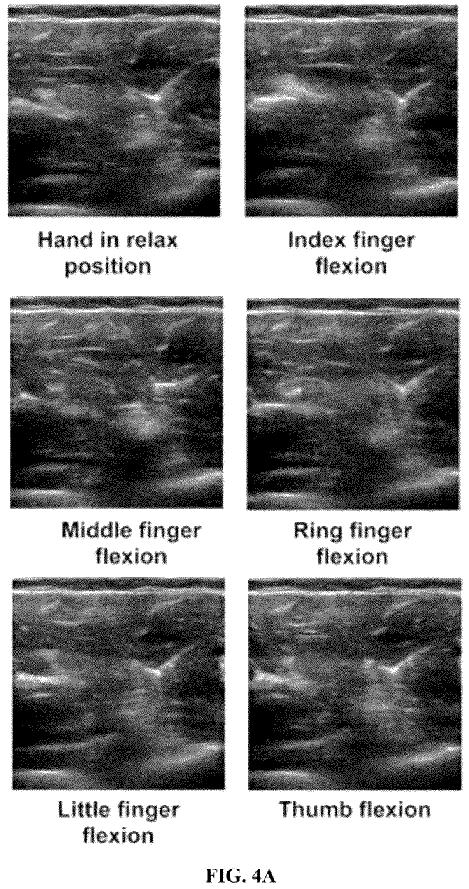

Since the musculoskeletal anatomy is different in the able-bodied and people with transradial limb loss, it is important to assess the accuracy of the proposed classification method for both groups. Therefore, the ultrasound images are separately obtained from the able-bodied group and the amputee group. In one example, each participant is asked to sit in a comfortable possession and put the hand on a cushion, as shown in FIG. 2. The palm is kept upwards. An ultrasound transducer 40, which is a palm-sized wireless ultrasound probe, is used to capture the muscle activities in different hand gestures. The ultrasound images of the main muscles responsible in finger flexion are evaluated. The ultrasound transducer 40 is a B-mode lightweight wireless ultrasound module with approximately 67 g, which is fixed on the forearm using a customized case. It is vital to note that the position and location of the ultrasound transducer 40 are critical in order to have greater control over the prosthetic hand device 100. The main muscles that perform different types of hand gestures and finger flexion are the flexor digitorum superficialis (FDS), flexor digitorum profundus (FDP), and flexor pollicis longus (FPL) muscles. To collect maximum muscle activities, the ultrasound transducer 40 is placed perpendicular (transverse) to the forearm at 30% to 50% of the length of the forearm from elbow, as indicated in FIG. 3.

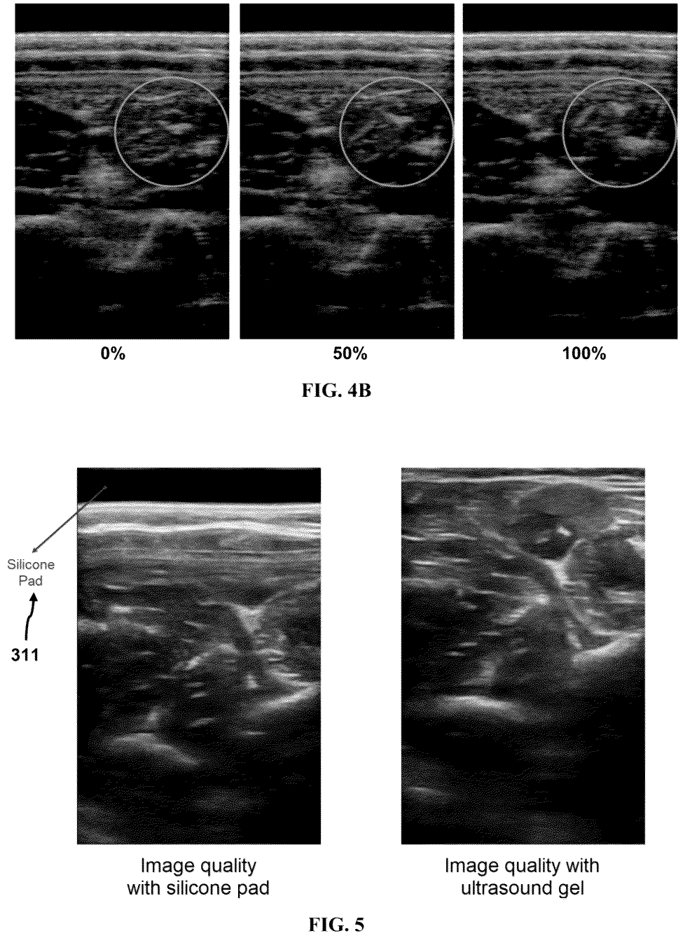

In the off-line test, both the able-bodied group and the amputee group are attended in this session and a plurality of hand gestures are studied, including rest, individual finger flexion (index, middle, ring, little and thumb), key pinch, fist, and pinch. It is apparent that the plurality of hand gestures may be otherwise without departing from the scope and spirit of the present disclosure. In one embodiment, each hand gesture is performed for 5 seconds and repeated for 5 times. To avoid fatigue and spasm in the muscles, a 15-second rest is provided between two hand gestures. For each finger position, plural images are captured and used for training, while some of the images are also used for validation. The B-Mode ultrasound images captured from muscle activities during performing different hand gestures are shown in FIG. 4A. In particular, the activities of the FDS, FDP, and FPL muscles are determined from the B-Mode ultrasound images.

In a further embodiment of the present disclosure, the prosthetic hand device 100 comprises a proportional control mechanism, as demonstrated by the ultrasound images in FIG. 4B. The purpose of the proportional control mechanism is to enable the amputee to dynamically control a speed of finger flexion and an angle of finger flexion in real-time with high precision. The proportional control mechanism is configured to monitor a degree of muscular contraction using the ultrasound images, thereby it is possible to classify different hand gestures from the ultrasound images captured and predict the proportional change of finger flexion. In certain embodiments, the muscular contraction ranges from 0% to 100%.

To improve the quality of the images and provide clearer visuals of the muscle activities, a gel pad or ultrasound gel is applied between the skin and the ultrasound transducer 40. However, using the gel pad or the ultrasound gel may create a problem. They can reduce the friction between the ultrasound transducer 40 and the skin, leading to misalignment and movement of the ultrasound transducer 40. This may result in a decrease in accuracy and reliability of the prosthetic control system. Moreover, prolonged exposure to moisture can cause damage to the skin, and there is a risk of contamination due to the ultrasound gel. To solve these problems, a custom-designed sticky silicone pad 311 was utilized. The sticky silicone pad 311 is made of biocompatible materials. The image quality of the ultrasound images using the sticky silicone pad 311 and ultrasound gel are compared in FIG. 5.

To create the sticky silicone pad 311, a molding technique may be used with biocompatible silicone liquid. Two different silicones with hardness ratings of Shore 00-00 and Shore 00-05 are used to create three different silicone pads. The first pad has a hardness of 00, which provided good resolution but is too sticky and difficult to put on the hand with the prosthesis. The second pad has a hardness of 05, which provided a good resolution for controlling the prosthesis, but was fragile and prone to damage during donning and doffing. The third pad is created by mixing the silicones with 00 and 05 hardness in a 3:1 ratio. Particularly, silicone liquid with hardness of 00 is mixed with another silicone liquid with hardness of 05 in a 3 to 1 ratio, and the mixed liquid is poured into a rectangular mold. The rectangular mold may have a thickness of between 0.5 mm to 1.5 mm, which defines the shape of the sticky silicone pad 311. The mixed liquid is solidified in the rectangular mold after keeping inside for 3 hours. Testing results shows that this sticky silicone pad 311 has a good image quality and is sticky enough to minimize transducer movement. In particular, the third pad is flexible enough to be used with a socket without causing any damage.

FIG. 6 shows the overall schematic of the classification process for extracting features to train a model that can classify different hand gestures in real-time. The transfer learning model 61 has a convolutional neural network (CNN) architecture selected from the group consisting of VGG16, VGG19, and Inceprion-Res-Net-V2. The CNN architectures of VGG16 and VGG19 have similar structures with 16 layers and 19 layers respectively. The Inceprion-Res-Net-V2 is a more complex architecture that combines the Inception architecture with the ResNet architecture to obtain a more efficient model. The weight and biases of the CNN architectures are pre-trained for extracting features from images. In one embodiment, the transfer learning model 61 has a VGG16 architecture that can achieve 92.7% top-5 test accuracy in ImageNet, which is an input dataset 61A of over 14 million images belonging to 1000 classes. In certain embodiments, the transfer learning model 61 comprises a plurality of convolutional layers 61B, a flatten layer 61C, and a fully connected layer 61D.

The plurality of convolutional layers 61B are the core building blocks of the transfer learning model 61, which are used for carrying out feature extraction through the application of convolution operations. Each convolutional layer comprises a set of kernel (or mask) that is convolved with the input image to produce a feature map. The flatten layer 61C performs flattening operation on the feature maps from the plurality of convolutional layers 61B to a one-dimensional vector. Lastly, the fully connected layer 61D is used to connect and combine the one-dimensional vectors from the flatten layer 61C for classification.

The ultrasound transducer 40 is placed perpendicular (transverse) to the forearm at 30% to 50% of the length of the forearm from elbow and captures ultrasound images of the FDS, FDP, and FPL muscles. The ultrasound images are collected from an able-bodied group, and an amputee group, which are separately obtained. In one embodiment, 70 ultrasound images are obtained for each hand gesture from each person and labelled accordingly. The ultrasound images are separated into two dataset groups: the first dataset group is a training dataset 65, and the second dataset group is a validation dataset 63. Since the ultrasound images collected cannot be processed by the AI model 60 directly, features should be extracted from the ultrasound images for determining the movements of the FDS, FDP, and FPL muscles. Particularly, the transfer learning model 61 is utilized to extract features from the training dataset 65 (first dataset group) to obtain the extracted features 62. The extracted features 62 are used to train a machine learning model 64 for classifying different hand gestures based on the ultrasound images. Therefore, the volitional movements of the amputee can be determined by classifying different hand gestures and individual finger flexions and extensions. On the other hand, the validation dataset 63 is utilized to evaluate an accuracy of the AI model 60. The machine learning model 64 comprises one or more machine learning algorithms selected from the group consisting of random forest (RF), k-nearest neighbors classifier (KNN), and support vector machine (SVM).

In certain embodiments, the ultrasound images contribute to the training dataset and the validation dataset to enhance the detection accuracy. Particularly, approximately 67% of the ultrasound images are used as the training dataset 65 and the remaining 33% of the ultrasound images are used as the validation dataset 63. It is apparent that the percentage of the ultrasound images may be otherwise without departing from the scope and spirit of the present invention. The classification accuracy (CA) may be calculated based on equation (1):

$$CA = \frac{number of correctly classified samples}{number of the whole validation samples} \times 100$$

Then, the accuracy of each machine learning algorithm is examined and compared. The above-described platform shows promising results in terms of the CA of the eight different hand gestures, which achieves 100% using different transfer learning methods and machine learning algorithms. Nevertheless, the time needed to train the model with different machine learning algorithms may vary.

The second embodiment of the present disclosure is related to the structure of the prosthetic device that can accurately replicate the function and movement of the normal muscle activity. More specifically, but without limitation, the present disclosure provides a prosthetic hand device 100 for a forearm amputee. One having ordinary skill in the art would understand that the current disclosure is also applicable to other prosthetic devices and exoskeleton devices, such as prosthetic knees and ankles.

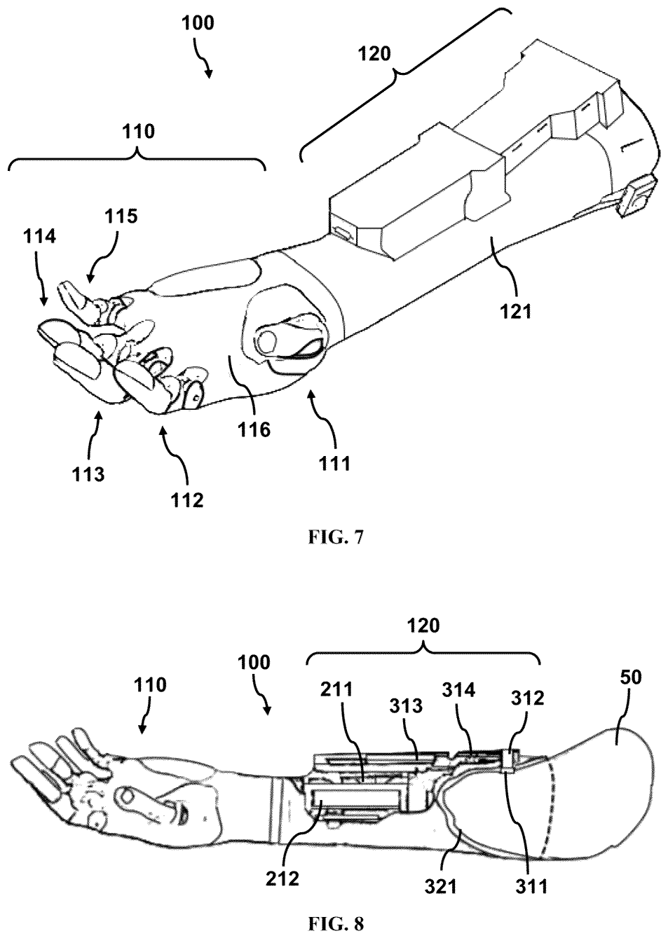

With reference to FIGS. 7-10, there is provided a prosthetic hand device 100 comprising a myoclectric hand 110 and a control assembly 120. The control assembly 120 is attached on a socket 121, wherein the socket 121 allows the prosthetic hand device 100 to be mounted on the residual limb 50 (or known as an arm stump) of an amputee. The shape of the socket 121 is based on a normal human hand, which may be scanned using a 3D scanner and printed with black nylon material using a 3D printer. The control assembly 120 is arranged to obtain ultrasound images of the residual limb 50 for controlling the prosthetic hand device 100 based on the volitional movement sensed. The myoelectric hand 110 is capable of performing hand motions including various hand gestures and individual finger flexions and extensions. The myoelectric hand 110 is connected to the socket 121 and becomes an extension of the forearm from the residual limb 50 of the amputee, which allows the amputee to perform a wide range of movements and actions. This can restore activities of the amputee and improve the quality of living and self-esteem.

The myoelectric hand 110 includes five mechanical fingers 111-115 and a base portion 116, which can be arranged in the form as shown in FIG. 7 with high biomimetic features that mimic the anatomy of a normal human hand. The five mechanical fingers 111-115 are actuatable to provide multiple degrees of freedom of movement. The base portion 116 is connected to and provides support to the five mechanical fingers 111-115. The first mechanical finger 111 is used as the thumb, the second mechanical finger 112 is used as the index finger, the third mechanical finger 113 is used as the middle finger, the fourth mechanical finger 114 is used as the ring finger, and the fifth mechanical finger 115 is used as the little finger. The second to fifth mechanical fingers 112-115 are parallel to each other and rotatable about a first axis A. The first mechanical finger 111 is rotatable about a second axis B other than and not parallel to the first axis A. The first mechanical finger 111 can perform abduction and adduction movement.

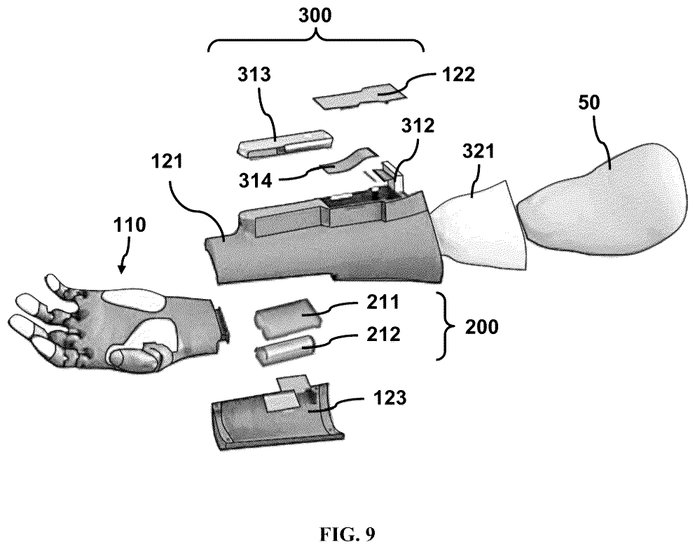

Human-machine interface (HMI) has been regulated via a variety of sensory modalities. In order to better comprehend the amputee's intended movements, sensing technologies for HMI have been created. With reference to FIG. 8 and FIG. 9, the control assembly 120 comprises a power module 200 having one or more batteries 212, and an ultrasound module 300 as an HMI. The ultrasound module 300 may be powered by a separated battery or the power module 200. In the illustrated embodiments, the ultrasound module 300 is provided on the anterior side of the socket 121, which can be dissembled by removing the top cover 122. The power module 200 is provided on the posterior side of the socket 121, which can also be dissembled by removing the bottom cover 123. It is apparent that the location and physical arrangement of the power module 200 and the ultrasound module 300 may be otherwise without departing from the scope and spirit of the present disclosure.

Advantageously, the present invention makes use of the ultrasound imaging, which can provide real-time dynamic images of interior tissue movements linked to physical and physiological activity, rather than the conventional sEMG sensors, for the prosthetic hand device 100. Ultrasound imaging allows a better discrimination between single motions or classification of full finger flexion, which provides a non-invasive and high precision method for controlling the prosthetic hand device 100 with multiple degrees of freedom. In certain embodiments, the ultrasound module 300 is configured to acquire ultrasound images of a region of the residual limb 50 for determining the type of hand gestures and individual finger flexions and extensions. In particular, the ultrasound module 300 is configured to capture the ultrasound images of the FDS muscle, the FDP muscle, and the FPL muscle. The power module 200 is configured to generate one or more output voltages necessary for driving the plural motors 603 of the prosthetic hand device 100. In one embodiment, the power module 200 comprises a battery management system 211 and one or more batteries 212. Preferably, the one or more batteries 212 are rechargeable battery cells.

In certain embodiments, the ultrasound module 300 includes an ultrasound transducer 312, a control circuit 313, a silicone pad 311, and a flexible cable 314 electrically connecting the control circuit 313 to the ultrasound transducer 312. The control circuit 313 is configured to cause the ultrasound transducer 312 to repeatedly and regularly generate acoustic waves which is directed into the residual limb 50 and propagate through the tissues, and then measure the acoustic reflections for the information to be used to generate the ultrasound images.

Since ultrasound gel or wet gel pad may be used to fill the gap between the skin and the ultrasound transducer 312 to collect the muscle activity with high resolution, such arrangement will cause skin problems and the ultrasound transducer 312 will be relocated due to low friction between the ultrasound transducer 312 and the skin. Further, it is not feasible to apply ultrasound gel between the skin and the ultrasound transducer 312 regularly. In view of the above, a silicone pad 311 is a biocompatible sticky silicone pad placed between the head of the ultrasound transducer 312 and the residual limb 50 for enhancing the image quality. As explained above, the silicone pad 311 is prepared by mixing the silicones with 00 hardness and 05 hardness in a 3:1 ratio. After using the silicone pad 311, the image quality of the ultrasound images is sufficiently good for controlling the prosthetic hand device 100, and it is sticky enough to minimize any movement of the ultrasound transducer 312. Additionally, the flexibility of the silicone pad 311 is good enough to be used with a liner 321 without any damage.

The primary point of contact between the prosthetic hand device 100 and the residual limb 50 is the liner 321, which is wrapped around the end of the residual limb 50 to create a suction that secures the prosthetic hand device 100 in place. To create the liner 321 that will securely attach the prosthetic hand device 100 to the amputee's residual limb 50, soft thermoplastic polyurethane (TPU) material with a shore A hardness of 50 is used. The use of the TPU material can reduce stress on the hand and make the liner 321 more comfortable for the amputee.

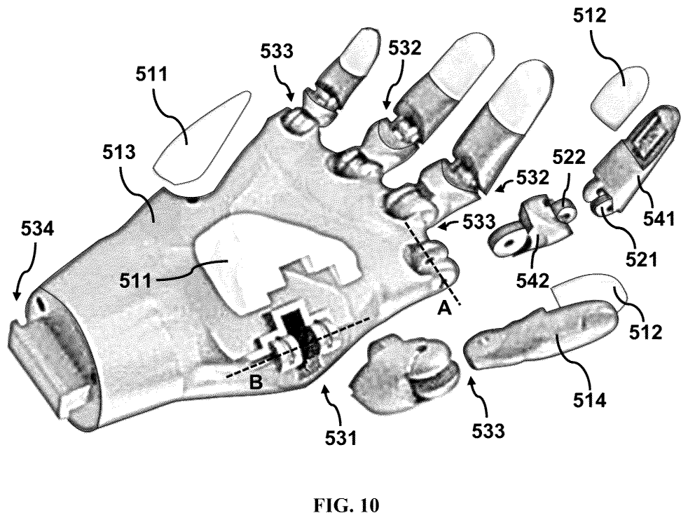

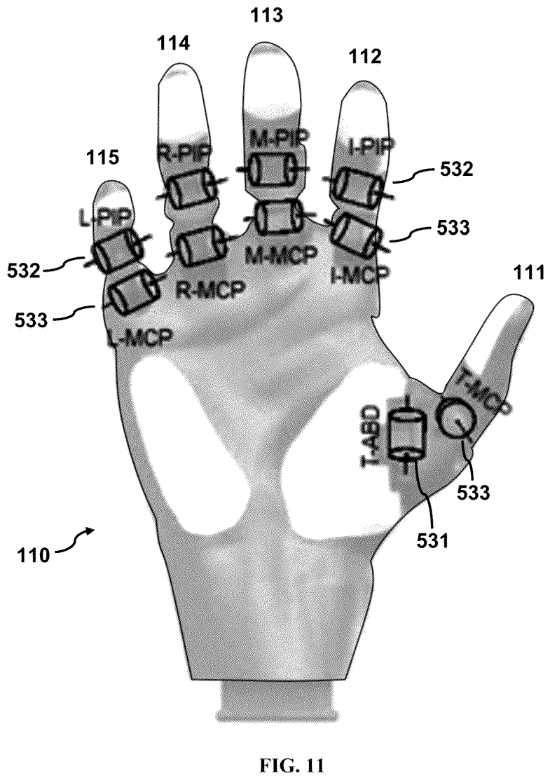

Referring to FIG. 10, each of the five mechanical fingers 111-115 and the base portion 116 are made of a base material 513, 514 and silicone 511, 512. In certain embodiments, the base material 513, 514 is selected from the group of materials consisting of nylon, plastic, polypropylene (PP), Acrylonitrile Butadiene Styrene (ABS), vinyl, and other suitable material with similar characteristics. The silicone 511, 512 has a frictional gripping characteristic with a shore hardness value of 00-50. The use of silicone 511, 512 can increase the friction between the objects and the myoelectric hand 110 and decrease the chance of slipping and falling objects from the myoclectric hand 110.

The myoelectric hand 110 has an actuating system to provide multiple degrees of freedom. The mechanical joints are positioned in the myoelectric hand 110 at locations based on a normal human hand, as conceptually illustrated in FIG. 11. In certain embodiments, the actuating system comprises plural artificial metacarpophalangeal (MCP) joints 533 at the five mechanical fingers 111-115, an additional MCP joint 531 at the first mechanical finger 111, and plural artificial proximal interphalangeal (PIP) joints 532 at the second to the fifth mechanical fingers 112-115. In certain embodiments, the additional MCP joint 531 is rotatable about the second axis B substantially orthogonal to the first axis A of the MCP joint 533 at the first mechanical finger 111. Therefore, the additional MCP joint 531 plays an important role in extending the mobility of the myoclectric hand 110, by allowing the first mechanical finger 111 to perform abduction and adduction. This can broaden the range of possible movements of the myoelectric hand 110, including grasping different types of objects and performing most of the daily living hand activities. In certain embodiments, the actuating system further comprises a wrist rotational joint 534 provided between the socket 121 and the myoelectric hand 110. The wrist rotational joint 534 is arranged to improve the dexterity, pinch, and wrist rotational movement of the myoelectric hand 110. Preferably, the prosthetic hand device 100 comprises an A-mode ultrasound transducer (not shown in the drawings) arranged to capture further ultrasound images for determining an intended wrist movement and controlling the wrist rotational joint 534. However, it is apparent that the prosthetic hand device 100 may comprises an sEMG to detect the intended wrist movement without departing from the scope and spirit of the present disclosure.

For each joint that allows rotation of a first finger element 541 about a second finger element 542, there is provided a pivotal pin (not shown) extended through a first mounting hole 521 on the first finger element 541 to a second mounting hole 522 on the second finger element 542.

FIG. 12 shows another exploded view of the myoelectric hand 110 for describing the actuating system. Different electrical components are mounted into the main body 410 of the myoclectric hand 110. In particular, the myoelectric hand 110 further comprises a Bluetooth module 431, a microprocessor 432, an artificial tendon 602, and a control unit 600 configured to actuate the artificial tendon 602 to flex and extend the five mechanical fingers 111-115.

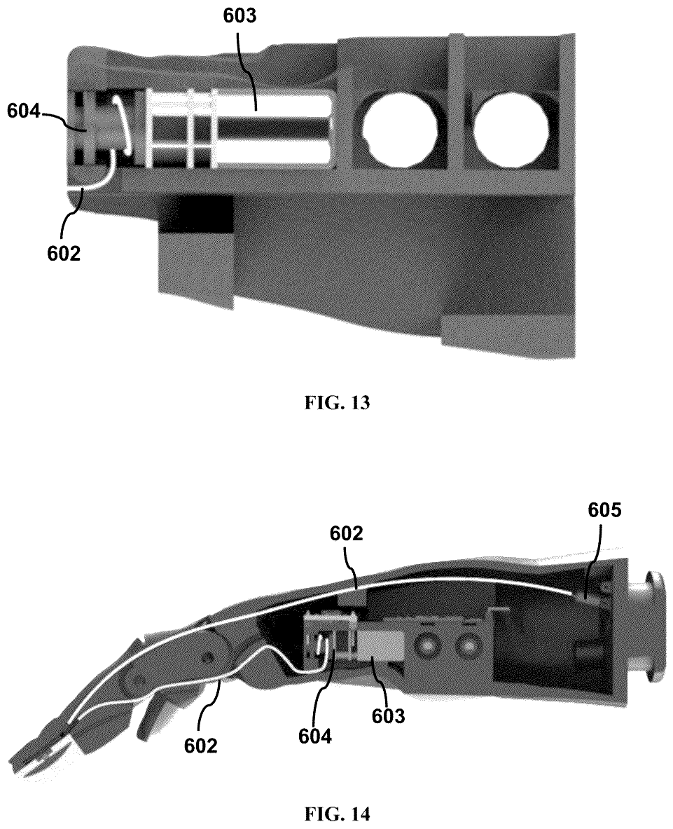

With reference to both FIGS. 12 to 14, the control unit 600 comprises a motor 603, a motor shaft 601, a roller 604, and a tension spring 605. The artificial tendon 602 is attached to the tension spring 605 at a first end, through the fingertip of an individual mechanical finger to the roller 604 at a second end. The roller 604 is fixed on the motor shaft 601. The artificial tendon 602 may be a fishing wire or other cables with sufficient durability. To flex each mechanical finger or perform thumb adduction movement, the motor 603 is powered to cause a motor shaft 601 and the roller 604 to rotate to drive a pulling movement of the mechanical finger via the artificial tendon 602, causing the mechanical finger to flex or adduct. The artificial tendon 602 is also connected to the tension spring 605 to allow finger extension and thumb abduction. The tension spring 605 stores the energy from flexion and releases the energy when the motor 603 is driven in an opposite direction, causing the mechanical finger to extend or to abduct.

In one embodiment, the ultrasound images obtained from the ultrasound module 300 of the prosthetic hand device 100 are sent to a computer system through Wi-Fi or other wireless communication interface. One or more processors of the computer system is configured to execute software instructions written using a computer programming language, such as Python, Java, JavaScript, or C++, to process the ultrasound images based on the AI model 60. The software instructions are programmed to perform extraction, training, and classification of the ultrasound images for determining the volitional movement of the amputee. The processor is then communicated with the prosthetic hand device 100 to transmit the predicted volitional movement, which is further sent to the microprocessor 432 via the Bluetooth module 431 or other wired or wireless communication devices. The microprocessor 432, based on the volitional movement, provides instructions to the control unit 600 to actuate the five mechanical fingers 111-115 dynamically and individually based on the volitional movement. It is apparent that the platform for processing the ultrasound images may also be provided in the prosthetic hand device 100, such that the volitional movement can be determined without connecting to an external system or using Wi-Fi. It is also possible that the platform is provided in the prosthetic hand device 100 and communicable with an external system using Wi-Fi, thereby the external system may from time to time provide training datasets 65 to train the machine learning model 64 for improving the accuracy.

According to the present disclosure, the prosthetic hand device 100 would struggle to pick up small objects if there is a lack of sensory feedback from the five mechanical fingers 111-115. The sensory feedback mechanism comprises plural sensors for collecting sensory information, including one or more temperature sensors, and plural force sensors. The sensory information is then conveyed to the amputee by stimulating the nerve, which allows the amputee to dynamically control a degree of flexion of each mechanical finger, and decrease the phantom pain. In certain embodiments, the sensory feedback mechanism is configured to transmit signals to the brain of the amputee by stimulating different nerves with different amplitudes and frequencies. Such nerve stimulation may be invasive, minimally invasive, or non-invasive.

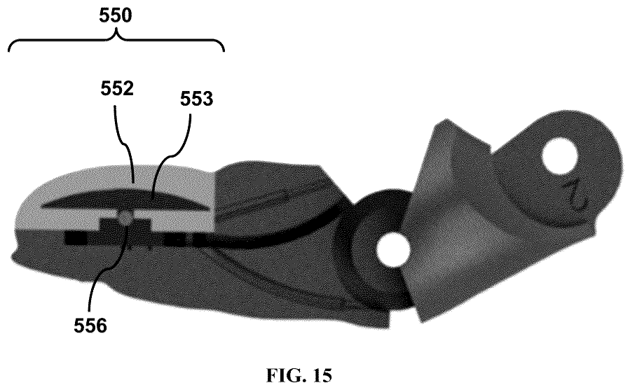

In certain embodiments, the force on each individual finger is determined as part of the sensory feedback mechanism. In order to control the amount of force provided by the prosthetic hand device 100, each of the five mechanical fingers 111-115 comprises a silicone layer 552 at a fingertip region 550, where the force sensor 556 is mounted in the fingertip region 550 under the silicone layer 552. The internal structure of the mechanical finger is shown in FIG. 15. The silicone layer 552 increases the friction between the mechanical finger and the objects. To transfer the applied force to the silicone layer 552, a curved surface part 553, made of black nylon material, ABS, or PP, was fixed under the silicone layer 552. The force can be measured by the force sensor 556 for understanding the fingertip force.

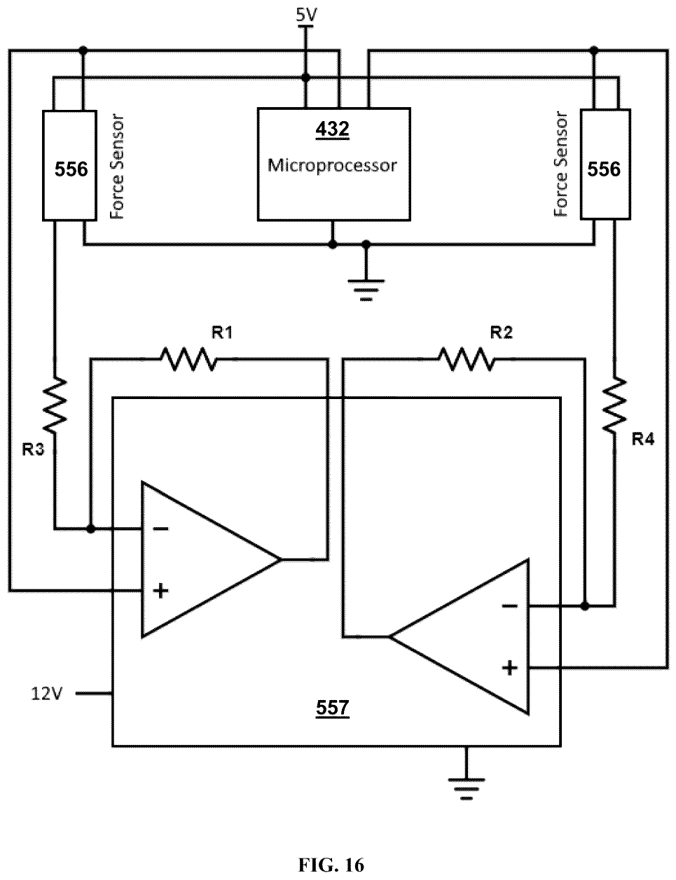

FIG. 16 shows a circuit diagram of the fingertip force measurement unit. Since the output voltage of the force sensor 556 is not high enough to be measured by microprocessor 432, an inverting amplifier is used to amplify the output voltage of the force sensor 556. In certain embodiments, a dual operational amplifier 557 is utilized to amplify the output voltages of two force sensors 556. The output of the microprocessor 432 serves as the input voltage for each force sensor 556, while each operational amplifier is powered separately by a battery or a power converter circuit. The output voltage after amplification is measured by the microprocessor 432 to convert the output of the force sensor 556 to the force applied to the fingertip. Therefore, the five mechanical fingers 111-115 can be dynamically controlled by the control unit 600 and the microprocessor 432 based on both the ultrasound images and the output voltages of the force sensors 556. In particular, by obtaining the output voltages of the force sensors 556 and transmitting the output voltages to the microprocessor 432, a more dedicated control of the five mechanical fingers 111-115 can be achieved.

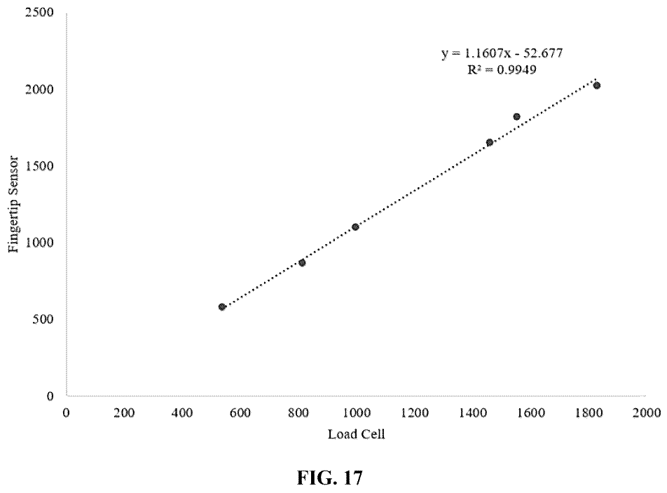

To assess the reliability of the force sensors 556, the value of the applied force is measured by the force sensors 556 mounted on the fingertip region 550 for different load cells. The result is shown in FIG. 17. The R-squared value and the mean square error (MSE) are 0.9844±0.0106 and 1399±458 grams respectively.

This illustrates a non-invasive and precise technique for controlling multiple degrees of freedom prosthetic hand device in accordance with the present disclosure. It will be apparent that variants of the above-disclosed and other features and functions, or alternatives thereof, may be integrated into other prosthetic devices for other body parts or exoskeleton devices. The present embodiment is, therefore, to be considered in all respects as illustrative and not restrictive. The scope of the disclosure is indicated by the appended claims rather than by the preceding description, and all changes that come within the meaning and range of equivalency of the claims are therefore intended to be embraced therein.

What is claimed is:

1. A prosthetic hand device mountable on a residual limb of an amputee, comprising:
a myoelectric hand comprising five mechanical fingers actuatable to provide multiple degrees of freedom of movement;
a control assembly comprising an ultrasound module as a human-machine interface (HMI), wherein the ultrasound module is configured to acquire ultrasound images of a region of the residual limb;
a transfer learning model having a convolutional neural network (CNN) architecture for obtaining extracted features from the ultrasound images; and
an artificial intelligence (AI) model executed by one or more processors and configured to classify the extracted features from the ultrasound images for determining a volitional movement of the amputee in real-time, and the volitional movement is transmitted to the myoelectric hand to dynamically and proportionally control the five mechanical fingers based on at least the volitional movement,
wherein:
the ultrasound module is configured to capture the ultrasound images of flexor digitorum superficialis (FDS), flexor digitorum profundus (FDP), and flexor pollicis longus (FPL) muscles for determining the volitional movement of the amputee.

2. The prosthetic hand device of claim 1 further comprises a proportional control mechanism enabling the amputee to dynamically control a speed of finger flexion and an angle of finger flexion, wherein the proportional control mechanism is configured to monitor a degree of muscular contraction using the ultrasound images for predicting a proportional change.

3. The prosthetic hand device of claim 1, wherein the transfer learning model further comprises a plurality of convolutional layers, a flatten layer, and a fully connected layer.

4. The prosthetic hand device of claim 1, wherein the ultrasound module comprises an ultrasound transducer and a control circuit, wherein:
the control circuit is configured to cause the ultrasound transducer to repeatedly and regularly generate acoustic waves which is directed into the residual limb of the amputee; and
the ultrasound transducer measures acoustic reflections for information to be used to generate the ultrasound images.

5. The prosthetic hand device of claim 4, wherein the ultrasound module further comprises a sticky silicone pad placed between head of the ultrasound transducer and the residual limb for enhancing image quality, wherein the sticky silicone pad is prepared by mixing silicones with 00 hardness and 05 hardness in a 3:1 ratio.

6. The prosthetic hand device of claim 1 further comprising a machine learning model, wherein:
the ultrasound images are separated into a training dataset and a validation dataset;
the transfer learning model extracts features from the training dataset to obtain the extracted features for training the machine learning model; and
the validation dataset is utilized to evaluate an accuracy of the AI model.

7. The prosthetic hand device of claim 6, wherein the machine learning model comprises one or more machine learning algorithms selected from the group consisting of random forest (RF), k-nearest neighbors classifier (KNN), and support vector machine (SVM).

8. The prosthetic hand device of claim 1, wherein the CNN architecture is selected from the group consisting of VGG16, VGG19, and Inceprion-Res-Net-V2.

9. The prosthetic hand device of claim 1, wherein the myoelectric hand comprises an actuating system to provide multiple degrees of freedom, wherein the actuating system comprises plural artificial metacarpophalangeal (MCP) joints at the five mechanical fingers, an additional MCP joint at the first mechanical finger, and plural artificial proximal interphalangeal (PIP) joints at the second to the fifth mechanical fingers, and wherein the additional MCP joint is rotatable about a second axis substantially orthogonal to a first axis of the MCP joint at the first mechanical finger to perform abduction and adduction.

10. The prosthetic hand device of claim 9, wherein the myoelectric hand comprises an artificial tendon and a control unit configured to actuate the artificial tendon to flex and extend an individual mechanical finger, wherein:
the control unit comprises a motor, a motor shaft, a roller, and a tension spring;
the artificial tendon is attached to the tension spring at a first end, through a fingertip of the individual mechanical finger to the roller at a second end;
the motor is powered to cause the motor shaft and the roller to rotate to drive a pulling movement of the individual mechanical finger via the artificial tendon to cause the individual mechanical finger to flex or adduct; and
the tension spring stores energy from flexion and releases the energy when the motor is driven in an opposite direction to cause the individual mechanical finger to extend or abduct.

11. The prosthetic hand device of claim 10, wherein:
the control assembly is attached on a socket having a shape based on a normal human hand; and
the actuating system further comprises a wrist rotational joint provided between the socket and the myoelectric hand, wherein the prosthetic hand device comprises an A-mode ultrasound transducer arranged to capture ultrasound images for determining an intended wrist movement and controlling the wrist rotational joint.

12. The prosthetic hand device of claim 1, wherein the myoelectric hand comprises a base portion connected to and provides support to the five mechanical fingers, and wherein:
each of the five mechanical fingers and the base portion are made of a base material and silicone;
the base material is selected from the group of materials consisting of nylon, plastic, polypropylene (PP), Acrylonitrile Butadiene Styrene (ABS), and vinyl; and
the silicone has a frictional gripping characteristic with a shore hardness value of 00-50.

13. The prosthetic hand device of claim 1 further comprising a sensory feedback mechanism comprising plural force sensors, wherein:
each of the five mechanical fingers comprises a silicone layer at a fingertip region, and the force sensor is mounted in the fingertip region under the silicone layer; and
the five mechanical fingers are dynamically and individually actuated by a control unit, which is controlled by a microprocessor based on the ultrasound images and output voltages of the force sensors.

14. The prosthetic hand device of claim 13, wherein the sensory feedback mechanism is configured to stimulating different nerves of the amputee with different amplitudes and frequencies to allow the amputee to dynamically control a degree of flexion of each of the five mechanical fingers, and decrease a phantom pain.

15. The prosthetic hand device of claim 13, wherein a curved surface part, made of black nylon material, ABS, or PP, is fixed under the silicone layer for transferring force to the force sensor.

16. A method for controlling a myoelectric hand using ultrasound images captured from a residual limb of an amputee, the method comprising:
acquiring, using an ultrasound transducer, acoustic reflections from a region of the residual limb for generating the ultrasound images that contribute to a training dataset and a validation dataset;
extracting, by a transfer learning model, features from the training dataset to obtain the extracted features, wherein the transfer learning model has a convolutional neural network (CNN) architecture;
performing, by one or more processors, real-time analysis on the extracted features for determining a volitional movement of the amputee;
transmitting the volitional movement to a microprocessor in the myoelectric hand comprising five mechanical fingers; and
providing instructions, by the microprocessor, to a control unit to actuate the five mechanical fingers dynamically and individually based on the volitional movement.

17. The method of claim 16, wherein the ultrasound transducer captures the ultrasound images of flexor digitorum superficialis (FDS), flexor digitorum profundus (FDP), and flexor pollicis longus (FPL) muscles for determining the volitional movement of the amputee.

18. The method of claim 16, wherein the step of performing real-time analysis on the extracted features for determining the volitional movement of the amputee further comprises training a machine learning model using the extracted features for classifying different hand gestures.

19. The method of claim 18, wherein the machine learning model comprises one or more machine learning algorithms selected from the group consisting of random forest (RF), k-nearest neighbors classifier (KNN), and support vector machine (SVM).

20. The method of claim 16 further comprising the step of causing the ultrasound transducer to repeatedly and regularly generate acoustic waves which is directed into the residual limb of the amputee.
